(19) United States
(12) Patent Application Publication
(10) Pub. No.: US 2015/0355170 A1
(43) Pub. Date: Dec. 10, 2015

(54) COMPOSITIONS, SYSTEMS AND METHODS THAT DETECT AND/OR REMOVE CROSS-REACTIVE ANTIBODIES FROM A BIOLOGICAL SAMPLE

(71) Applicant: BHC Technology Holdings LLC, ST. PAUL, MN (US)

(72) Inventor: Daniel Patrick Collins, Lino Lakes, MN (US)

(73) Assignee: BHC TECHNOLOGY HOLDINGS LLC, ST. PAUL, MN (US)

(21) Appl. No.: 14/739,677

(22) Filed: Jun. 15, 2015

Related U.S. Application Data
(62) Division of application No. 14/039,222, filed on Sep. 27, 2013.
(60) Provisional application No. 61/708,136, filed on Oct. 1, 2012.

(51) Int. Cl. G01N 33/53

(52) U.S. Cl. CPC G01N 33/5306

(57) ABSTRACT

The present invention generally relates to compositions, systems and methods that detect and/or remove cross-reactive antibodies from a biological sample. In many cases, the cross-reactive antibodies are human anti-animal antibodies. In certain cases, the cross-reactive antibodies are human anti-mouse antibodies.

### RELATED APPLICATIONS

This application claims priority to U.S. application Ser. No. 14/039,222, which in turn claims priority to U.S. provisional application No. 61/708,136, the entire contents of which are incorporated herein by reference.

### FIELD OF THE INVENTION

The present invention generally relates to compositions, systems and methods that detect and/or remove cross-reactive antibodies from a biological sample.

### BACKGROUND OF THE INVENTION

Immunoassays are commonly used for detecting levels of an analyte that give diagnostic information. Typically, a biological sample is collected and used in an immunoassay to detect levels of an analyte that indicate a variety of different conditions ranging from autoimmune diseases and infectious diseases.

Many immunoassays use monoclonal antibodies that target a particular antigen. Monoclonal antibodies are typically made by fusing myeloma cells with spleen cells from an animal immunized with a desired antigen. The fused cell then secretes antibodies that are collected and used in immunoassays. These animal antibodies are often mouse, rabbit, goat, sheep, cow, pig, rate and horse antibodies. The most common animal antibody is a murine or mouse antibody.

Not only are animal antibodies used in immunoassays, they are also used in monoclonal antibody therapies to treat a variety of conditions ranging from cancer, cardiovascular disease, inflammatory diseases, autoimmune diseases, macular degeneration, transplant rejection, multiple sclerosis and viral infection. The monoclonal antibodies are injected into a patient's body and help the patient's immune system in fighting the patient's condition.

Patients receiving monoclonal antibody therapies are repeatedly exposed to these animal antibodies. Many immune systems treat animal antibodies as foreign proteins and in turn produce new human antibodies that target the animal antibodies. These new human antibodies are often called human anti-animal antibodies (“HAAA”). Because the most common animal antibody used in therapies is the mouse antibody, the most common HAAA is the human anti-mouse antibody (“HAMA”). Researchers have determined that an increasing percentage of the general human population include HAAAs, particularly HAMAs.

The increase in HAAAs has presented a number of problems for immunoassays. In a standard sandwich immunoassay, a substrate is provided that includes bound monoclonal animal antibodies. A biological sample is then contacted with the substrate. If the target antigen is in the biological sample, the bound monoclonal animal antibodies bind to an epitope on the antigen. Next, labeled detection antibodies are introduced that bind to another epitope on the antigen. Finally, a detection process is used to detect the number of labeled detecting antibodies, which correlates to the number of the antigen.

HAAAs often go unnoticed and produce false positives and false negatives in immunoassays. In certain cases, HAAAs can cause false positives. When the biological sample is contacted with the substrate, if the sample includes HAAAs, the HAAAs bind to the monoclonal animal antibodies on the substrate. Also, the labeled detection antibodies also bind to the HAAAs. This can result in cross-binding of the substrate-bound capture antibody and the labeled detection antibody even in the absence of antigen. Here, the detect process detects the presence of the labeled animal antibodies, which falsely correlates to the presence of the antigen and generates a false positive result.

In other cases, HAAAs can cause false negatives in immunoassays. When the biological sample is contacted with the substrate, the monoclonal animal antibodies on the substrate bind to HAAAs rather than the target antigen thus blocking by direct interference with the binding site or by steric interference. Also, many of the labeled detection antibodies bind to free HAAAs in the sample and are inhibited in binding to the antigen for the same reasons as the substrate-bound capture antibody. After the biological sample is separated from the substrate, labeled detection antibodies bound to free HAAAs are also separated from the substrate. Here, the detection process detects less of the labeled detection antibodies, which falsely correlates to the absence of the antigen and generates a false negative result. Such false positive and false negative results confuse diagnosis and either provoke therapy where it is not needed or deny therapy where it is warranted.

It would be desirable to detect the presence of cross-reactive antibodies in a biological sample. It would also be desirable to remove cross-reactive antibodies from a biological sample before using the biological sample in an immunoassay.

### SUMMARY

Certain embodiments provide a capture system that captures a desired cross-reactive antibody in a biological sample. The capture system includes a target particle and a target antibody, wherein the target antibody is bound to the target particle and binds to the desired cross-reactive antibody in the biological sample.

Other embodiments provide a composition that includes a suspension fluid and a plurality of capture systems suspended in the suspension fluid, wherein each capture system in the plurality of capture systems comprises a target particle and a target antibody, wherein the target antibody is bound to the target particle and binds to a desired cross-reactive antibody in a biological sample.

Other embodiments provide a method of removing a cross-reactive antibody from a biological sample. The removal method includes steps of (a) providing capture systems each comprising a target particle and a target antibody, wherein the target antibody is bound to the target particle, (b) providing a biological sample, wherein the biological sample comprises a cross-reactive antibody, (c) adding the capture systems to the biological sample, wherein the target antibody binds to the cross-reactive antibody, and (d) removing the capture systems from the biological sample, wherein the cross-reactive antibody is bound to the target antibody and the target antibody is bound to the target particle.

Other embodiments provide for a method including steps of (a) providing an immunoassay that uses an immunoassay antibody, (b) providing a capture system comprising a target particle and a target antibody, wherein the target antibody is bound to the target particle, (c) providing a biological sample, wherein the biological sample comprises a cross-reactive antibody that reacts with the immunoassay antibody, (d) adding the capture system to the biological sample, wherein the target antibody binds to the cross-reactive antibody, (e) removing the capture system from the biological sample, wherein the removing the capture system also removes the cross-reactive antibody bound to the target antibody and wherein the biological sample becomes substantially free of the cross-reactive antibody and (f) using the biological sample in an immunoassay.

Other embodiments provide a method of identifying a cross-reactive antibody from a biological sample. The method can include steps of (a) providing a capture system, the capture system comprising a target particle and a target antibody, wherein the target antibody is bound to the target particle, (b) providing a biological sample, wherein the biological sample comprises a cross-reactive antibody, (c) adding the capture system to the biological sample, wherein the target antibody binds to the cross-reactive antibody, (d) removing the capture system from the biological sample, wherein the cross-reactive antibody is bound to the target antibody and the target antibody is bound to the target particle, (e) mixing a detection antibody with the capture system, wherein the detection antibody binds to the cross-reactive antibody and (f) analyzing the capture system to detect the detection antibody, wherein the detection antibody is bound to the cross-reactive antibody and the cross-reactive antibody is bound to the target antibody and the target antibody is bound to the target particle, and wherein detection of the detection antibody indicates presence of the cross-reactive antibody.

Other embodiments provide an immunoassay kit that includes an immunoassay that uses an immunoassay antibody and a capture system that captures a cross-reactive antibody in a biological sample, wherein the cross-reactive antibody reacts with the immunoassay antibody, wherein the capture system comprises a target particle and a target antibody, wherein the target antibody is bound to the target particle and binds to the cross-reactive antibody in the biological sample.

In some embodiments, the target antibody is an animal antibody and the desired cross-reactive antibody is a human anti-animal antibody. For example, the target antibody can be an animal antibody obtained from an animal selected from the group consisting of mouse, pig, hamster, rat, sheep, goat, horse, cow and chicken. In some cases, the target antibody is a mouse antibody and the desired cross-reactive antibody is a human anti-mouse antibody. The target antibody can also have an isotype selected from the group consisting of IgG1, IgG2a, IgG2b, IgG3, 4, IgM, IgA, IgE, IgY, kappa and lambda isotypes.

In some embodiments, the target antibody is of the same isotype or of the same species as the immunoassay antibody. In certain cases, the target antibody is the same as the immunoassay antibody. In some embodiments, the target antibody and the immunoassay antibody are animal antibodies and the cross-reactive antibody is a human anti-animal antibody. In certain cases, the target antibody and the immunoassay antibody are mouse antibodies and the cross-reactive antibody is a human anti-mouse antibody.

The capture system can also include a target particle coated with avidin or streptavidin and a target antibody labeled with biotin, wherein the avidin or streptavidin binds to biotin. Some embodiments of the invention provide a method of preparing a composition that removes cross-reactive antibodies from a biological sample. The method includes steps of (a) coating a target particle with avidin or streptavidin, (b) labeling a target antibody with biotin, (c) mixing the target particle with the target antibody for a period of time to allow the avidin or streptavidin to bind to the biotin, which causes the target particle to bind to the target antibody, and (d) suspending the bound target particle and target antibody in a suspension fluid.

### BRIEF DESCRIPTION OF THE DRAWINGS

The following drawings are illustrative of particular embodiments of the invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is a schematic representation of a capture system that captures a cross-reactive antibody in a biological sample.

FIG. 2 is a schematic representation of a detection system that detects a heavy-chain specific cross-reactive antibody in a biological sample.

FIG. 3 is a schematic representation of a detection system that detects a light-chain specific cross-reactive antibody in a biological sample.

FIG. 4 is a schematic representation of a detection system that detects an idiotype specific cross-reactive antibody in a biological sample.

FIG. 5 is a flow-chart of a method of removing cross-reactive antibodies from a biological sample.

FIG. 6 is a scatter plot showing a light scatter pattern of paramagnetic particles.

FIG. 7 is a single parameter histogram showing identification of human anti-mouse OKT-3 cross-reactive antibody by capture with a mouse monoclonal OKT-3 target antibody and detection with phycoerythrin-labeled anti-human lambda light chain specific antibody.

FIG. 8 is a single parameter histogram showing identification of human anti-mouse OKT-3 cross-reactive antibody by capture with a mouse monoclonal OKT-3 target antibody and detection with phycoerythrin-labeled anti-human kappa light chain specific antibody.

FIG. 9 is a single parameter histogram showing identification of human anti-mouse OKT-3 cross-reactive antibody by capture with a mouse monoclonal OKT-3 target antibody and detection with cy5-phycoerythrin-labeled anti-human heavy chain specific antibody.

FIG. 10 is a single parameter histogram showing non-detection of human anti-mouse OKT-3 cross-reactive antibody with a mouse monoclonal IgM target antibody and cy5-phycoerythrin-labeled anti-human heavy chain specific antibody.

FIG. 11 is a single parameter histogram showing non-detection of human anti-mouse OKT-3 cross-reactive antibody by a mouse monoclonal IgG1 target antibody and cy5-phycoerythrin-labeled anti-human heavy chain specific antibody.

FIG. 12 is a single parameter histogram showing an assay of human anti-mouse OKT-3 cross-reactive antibody by capture with a mouse monoclonal OKT-3 target antibody and detection with phycoerythrin-labeled anti-human lambda specific antibody.

FIG. 13 is a single parameter histogram showing an assay of human anti-mouse OKT-3 cross-reactive antibody by capture with a mouse monoclonal OKT-3 target antibody and detection with phycoerythrin-labeled anti-human lambda specific antibody.

### DETAILED DESCRIPTION

The present invention relates to compositions, systems and methods for detecting and/or removing a cross-reactive antibody from a biological sample. Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. Publications, patent applications, patents, and other references mentioned herein are incorporated by reference. In case of conflict, the present specification, including definitions, will control. In addition, the materials, methods, and examples are illustrative only and not intended to be limiting.

Certain embodiments provide for a capture system that captures a cross-reactive antibody in a biological sample. As used herein, the term “biological sample” refers to any biological sample, whether human or non-human, and includes but is not limited to fluids and tissue samples. In certain cases, the biological sample is a fluid such as serum, plasma, whole blood, tissue fluid and urine.

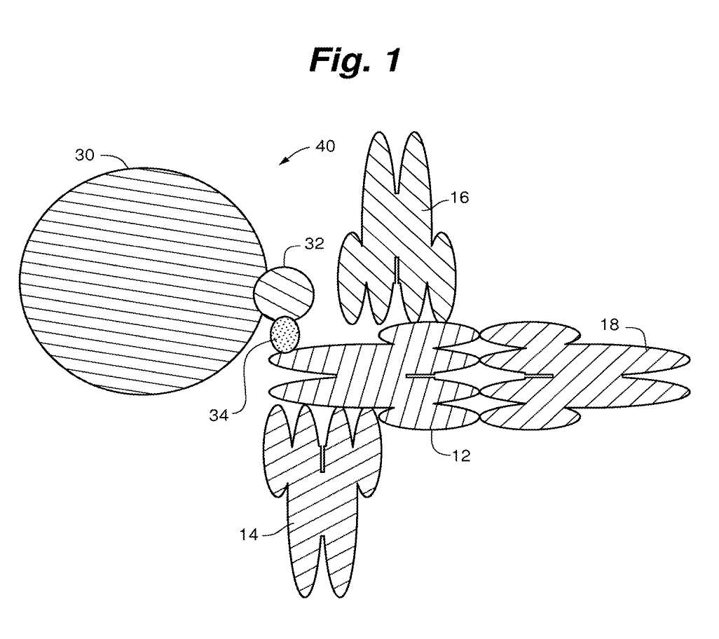

FIG. 1 illustrates a capture system 100 according to one embodiment. The capture system 100 includes a target particle 30 bound to a target antibody 12. As used herein, the term “target particle” refers to any solid-phase particle. The target particle 30 can be a microscopic particle. In certain cases, the target particle 30 has a diameter of between 10 nanometers to 200 microns. In one embodiment, the solid-phase particle 30 has a diameter of between about 1 microns to about 10 microns, such as 7 microns.

The target particle 30 can also have any desired shape. In some cases, the target particle 30 has a spheroidal shape. Also, in some cases, the target particle 30 is a paramagnetic particle. As used herein, the term “paramagnetic particle” refers to a particle that has paramagnetic properties in the presence of a magnetic field. In the presence of a magnetic field, the paramagnetic particles become magnetized and move through a liquid medium. In certain cases, the paramagnetic particle consists of ferric and/or ferrous oxides coated with plastics like polystyrene or other coatings like collagen, cellulose that confine the paramagnetic material into a particle and can also serve as chemically modifiable surfaces to attach proteins like antibodies, avidin, and streptavidin.

The target antibody 12 is bound to the target particle 30. In one embodiment, the target particle 30 is coated with avidin or streptavidin 32 and the target antibody 12 is labeled with biotin 34. Target antibody 12 labeled with biotin 34 is also referred to as a biotinylated antibody. Biotinylated antibodies can either be purchased commercially or be created using standard methods. One exemplary method of creating biotinylated antibodies is described in Example 8 below. Avidin or streptavidin coated solid-phase particles are available from a number of commercial sources. In one embodiment, the target particle 30 is a paramagnetic magnetic particle coated with avidin 32 obtained from Spherotech in Libertyville, Ill.

In certain embodiments, the target particle 30 is a particle coated with avidin 32 and target antibody 12 is a biotinylated antibody labeled with biotin 34. The avidin 32 binds to the biotin 34, thereby binding the target antibody 12 to the target particle 30. Avidin/streptavidin binding to biotin is advantageous because it has a very high affinity (ka˜10¹⁵), which is almost as strong as covalent bonding. This strong bonding helps to ensure that the target antibody 12 remains bound to the target particle 30 during detecting and/or removing processes.

The target antibody 12 is an antibody that binds to a cross-reactive antibody in a biological sample. As used herein, the term “cross-reactive antibody” refers to any antibody in a biological sample that undesirably binds to an immunoassay antibody. As used herein, the term “immunoassay antibody” refers to any antibody in an immunoassay.

In some embodiments, the target antibody 12 is of the same species as the immunoassay antibody. Common immunoassay antibodies include but are not limited to mouse, guinea pig, hamster, rat, sheep, goat, horse, cow and chicken antibodies. In certain cases, the immunoassay antibody is a mouse antibody. In particular cases, the immunoassay antibody is a mouse monoclonal antibody. Thus, the target antibody 12 can be an animal antibody. In certain embodiments, the target antibody 12 is an animal antibody obtained from an animal source selected from the group consisting of mouse, pig, hamster, rat, sheep, goat, horse, cow and chicken. In certain cases, the target antibody 12 is a mouse antibody.

The target antibody 12 can also be a monoclonal antibody or a polyclonal antibody. In certain cases, the target antibody 12 is a monoclonal antibody. In particular cases, the target antibody 12 is an animal monoclonal antibody, such as a mouse monoclonal antibody. Suitable animal monoclonal antibodies are available from a variety of commercial sources including Becton Dickinson, Beckman coulter, Biospacific and others. Suitable monoclonal antibodies can also be prepared using standard hybridoma technologies known in the art. In one embodiment the target antibody 12 is an OKT3 mouse monoclonal antibody.

In other embodiments, the target antibody 12 is of the same isotype as the immunoassay antibody. Common immunoassay antibody isotypes include but are not limited to heavy chain isotypes such as IgG1, IgG2a, IgG2b, IgG3, IgG4, IgM, IgA, IgE and IgY isotypes and light chain isotypes such as kappa and lambda isotypes. Thus, the target antibody 12 can be an antibody having a heavy chain isotype selected from the group consisting of IgG, IgM, IgE, IgA, IgD, IgY and any subclass thereof. In certain embodiments, the heavy chain isotype is an IgG isotype selected from the group consisting of IgG1, IgG2a, IgG2b, IgG3, and IgG4 subclasses. In other embodiments, the target antibody 12 has a light chain isotype selected from the group consisting of kappa and lambda isotypes. In certain embodiments, the target antibody 12 has an isotype directed against keyhole limpet hemcyanin.

Additionally, in certain cases, the target antibody 12 has the same specificity as the immunoassay antibody. Common immunoassay specificities include but are not limited to OKT3 (CD3), herceptin and rituximab specificities. Thus, in certain embodiments, the target antibody 12 has an idiotype selected from the group consisting of OKT3 (CD3), herceptin and rituximab. In one embodiment, the target antibody 12 has an OKT3 (CD3) idiotype.

In many cases, the target antibody 12 is the same antibody as the immunoassay antibody. For example, if the immunoassay antibody is a monoclonal mouse antibody, the target antibody 12 can be the same monoclonal mouse antibody. In one example, the immunoassay antibody is a monoclonal mouse IgG1 antibody and the target antibody 12 is the same monoclonal mouse IgG1 antibody. In other example, the immunoassay antibody is a monoclonal mouse IgM antibody and the target antibody 12 is the same monoclonal mouse IgM antibody. In another example, the immunoassay antibody is a monoclonal mouse OKT3 antibody and the target antibody 12 is the same monoclonal mouse OKT3 antibody.

The target antibody 12 is also an antibody that reacts with a cross-reactive antibody in a biological sample. Cross-reactive antibodies will bind to a target antibody 12 through three major sites: a heavy chain reactive site, a light chain reactive site and an idiotype reactive site. As shown in FIG. 1, a heavy chain-specific cross-reactive antibody 14 binds to the heavy chain reactive site, a light chain-specific cross-reactive antibody 16 binds to the light chain reactive site and an idiotype-specific cross-reactive antibody 18 binds to the idiotype reactive site.

In many cases, the target antibody 12 is an animal antibody and the cross-reactive antibody is a HAAA. In certain cases, the target antibody 12 is a mouse antibody and the cross-reactive antibody is a HAMA. Also, in some cases, the target antibody 12 is a pathogen-specific antibody and the cross-reactive antibody is a human anti-pathogen-specific antibody. In certain cases, the target antibody 12 is a monoclonal mouse OKT3 antibody and the cross-reactive antibody is a human anti-mouse OKT3 antibody.

Some embodiments of the invention also provide for a composition that includes a plurality of the capture systems 40 described above (“target composition”). The capture system 40 in the target composition can have any of the embodiments described above. In some cases, the target composition includes capture systems 40 each having the same target antibody 12. Here, the target composition includes capture systems 40 all having a singular species origin, a singular isotype and/or a singular specificity.

In other embodiments, the target composition includes capture systems 40 having a plurality of different target antibodies 12. In other words, the different target antibodies 12 have different species origins, different isotypes and/or different specificities. Here, the target composition includes capture systems 40 having plural species origins, plural isotypes and/or plural specificities. In some cases, the target composition includes capture systems 40 wherein the target antibodies 12 include all antibodies used in a desired immunoassay.

Target compositions that have a plurality of capture systems 40 with different target antibodies 12 are advantageous because they allow for creation of a flexible capture system that can be used to capture a variety of cross-reactive antibodies in a biological sample. Such a highly flexible system can be used to capture and remove cross-reactive antibodies in a biological sample before using the biological sample in a large plurality of immunoassays. This system allows for development of a “universal” cross-reactive antibody capture and removal systems.

The target composition includes capture systems 40 in an amount sufficient to remove a cross-reactive antibody from a biological sample. Again, as used herein the term “capture system” includes a target particle 30 bound to a target antibody 12. In some embodiments, the target composition includes capture systems 40 suspended in a suspension fluid, for example phosphate-buffered saline (“PBS”). In some cases, the target composition includes target antibodies 12 labeled with biotin 34 and bound to paramagnetic particles coated with avidin 32 and suspended in PBS. In some cases, the target composition includes capture systems 40 suspended in PBS in a concentration of 30,000 to 100 million target particles/ml. In certain cases, the target composition includes capture systems 40 suspended in PBS at a concentration of 3 million target particles/ml.

Other embodiments provide for a method of removing a cross-reactive antibody from a biological sample. The method includes steps of providing capture systems 40 and providing a biological sample 80. The capture systems 40 can be capture systems according to any of the embodiments described above.

The method also includes adding or mixing the capture systems 40 to the biological sample 80. In some cases, the method includes mixing the capture systems 40 with the biological sample for a period of time sufficient to allow binding of cross-reactive antibodies to target antibodies 12. In many cases, the period of time is between about 5 minutes and about 2 hours. In some cases, the period of time is between about 15 minutes and about 30 minutes. The method can also include subjecting the mixture to conditions that maximize the binding of cross-reactive antibodies to the target antibodies 12. In some cases, the mixture is mixed for a period of time using an orbital mixer. In other cases, the mixture is incubated for a period of time.

After this period of time expires, the method includes removing the capture systems 40 (along with any cross-reactive antibody bound to the target antibody 12) from the biological sample. In some cases, the method includes centrifuging the mixture to separate the capture systems 40. In other cases, the target particles 30 include paramagnetic particles and the method includes magnetically sequestrating the mixture 90 to separate the capture systems 40. The use of paramagnetic particles are advantageous because they are easier to visualize and are easier to remove using a magnetic field.

Once the capture systems 40 are separated, the biological sample can be removed or extracted for later use in an immunoassay. The removed biological 95 sample no longer has significant cross-reactive antibodies. The absence of these cross-reactive antibodies ensures that the immunoassay will not have a false negative or false positive caused by cross-reactive antibodies. Also, the biological sample 95 has not been diluted. Further, the biological sample 95 does not contain any other material ingredients added (e.g., competitive antibodies). This removal system is a significant improvement over current dilutative systems of removing cross-reactive antibodies, which often render the biological sample too diluted to be usable in an immunoassay. Further, this removal system does not add competitive antibodies to the biological sample, such competitive antibodies often causes additional cross-reactive antibodies to interfere in an immunoassay. This removal system is also a highly specific removal system that directly targets the cross-reactive antibody desired to be removed.

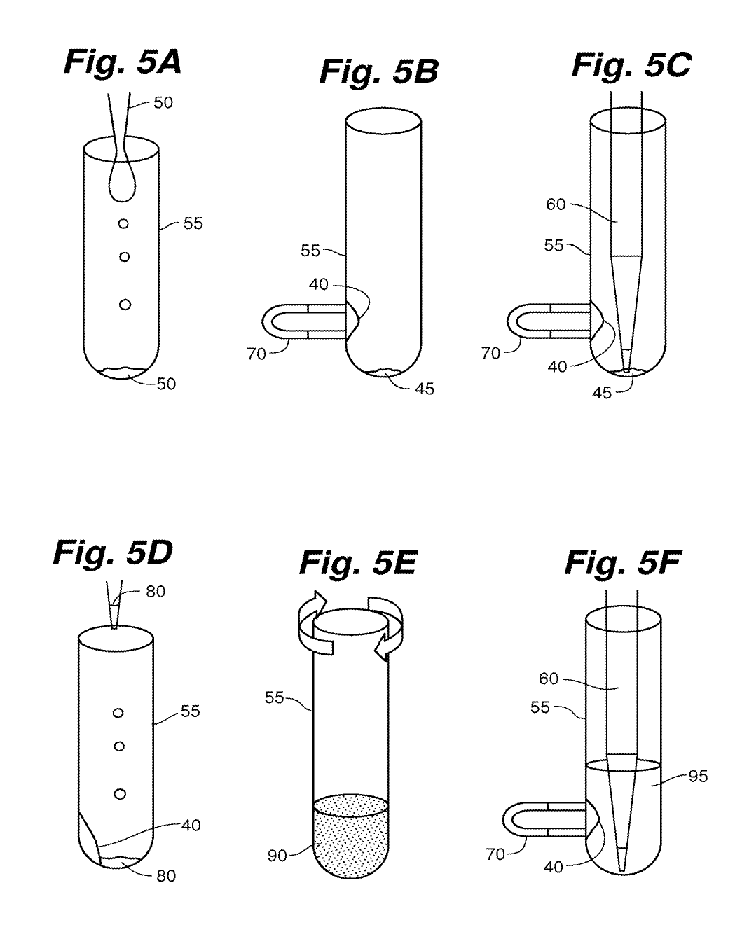

One exemplary removal method will now be described with reference to FIG. 5. FIG. 5 illustrates steps A through F. At step A, a target composition 50 is added to a container, e.g., a test tube 55. The target composition 50 includes capture systems 40 with paramagnetic particles 30 suspended in a suspension liquid 45. The target composition 50 is added in an amount of 10 microliters of target composition per 100 microliters of biological sample to be added.

At step B, the test tube 55 is placed in a magnetic field with a magnet 70. The magnetic field causes the paramagnetic particles 30 in the capture systems 40 to separate the capture systems 40 from the suspension liquid 45. At step C, the suspension liquid 45 is extracted with a pipette 60. At step D, a biological sample 80 is added to the test tube 55. At step E, the biological sample 80 mixes with the capture systems 40 to form a mixture 90. The test tube 55 is also placed in an orbital mixture for 15 minutes to thoroughly mix the mixture 90 and to maximize binding of target antibodies 12 to cross-reactive antibodies.

At step F, the test tube 55 is again placed in a magnetic field with a magnet 70. The magnetic field causes the paramagnetic particles 30 in the capture system 40 (and cross-reactive antibodies bound to the capture system 40) to separate from the biological sample. The separated biological sample 95 is substantially free of cross-reactive antibodies. This biological sample 95 is also extracted with a pipette and is now ready to be used in an immunoassay.

The current removal system is advantageous because the mixing of capture systems 40 throughout the biological sample 80 allows for more surface area of the biological sample 80 to come in contact with the target antibodies 12. In previous systems, a limited volume of biological sample is added to a substrate that contains antibodies. Less surface area of the biological sample contacts the substrate. Thus, the current capture system has an increased binding capacity over previous systems.

In some embodiments, the removal method includes providing a biological sample 80 in any desired sample container and adding capture systems 40 directly to the sample container. More or less capture systems 40 can be added depending on the volume of the biological sample 80 and the volume of the sample container. The capture systems 40 then mix throughout the biological sample 80 and form a mixture 90. In certain cases, method also includes a step of mixing the mixture 90 or incubating the mixture 90 for a period of time. The capture systems 40 (and cross-reactive antibodies bound to the capture system 40) are then separated from the biological sample and removed. This embodiment is advantageous because the capture systems can be added to any desired container containing any desired volume of biological sample.

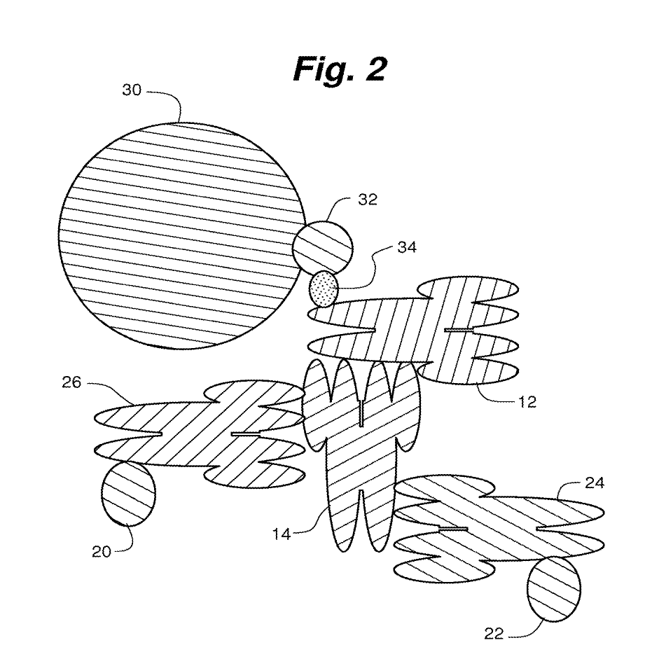

Other embodiments of the invention provide for a detection system that detects and identifies a cross-reactive antibody in a biological sample. FIGS. 2-4 illustrate detection systems according to certain embodiments. In each of these embodiments, the detection system also includes the capture system 40 described above.

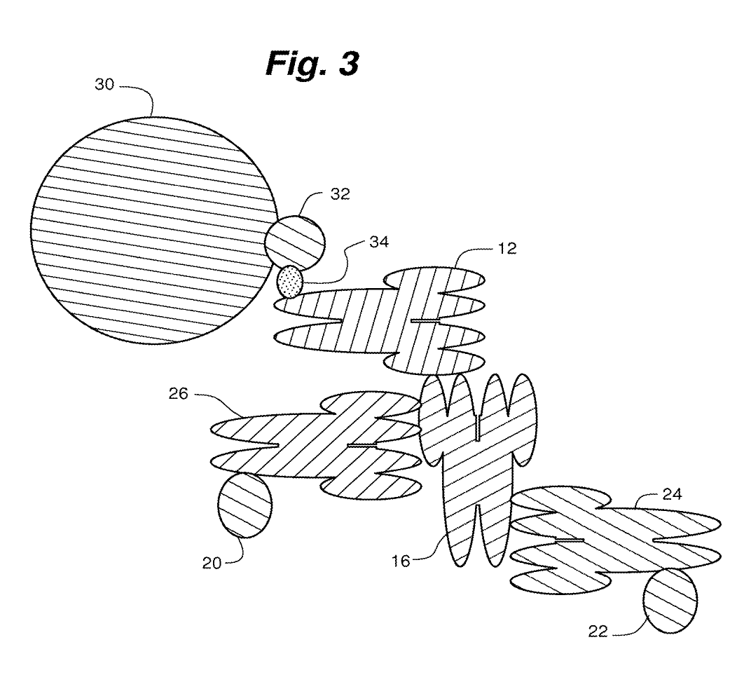

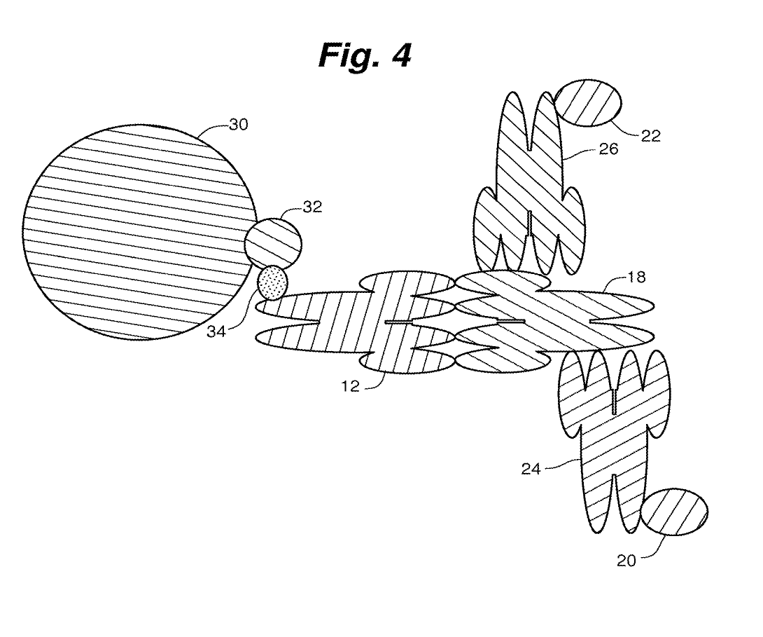

FIG. 2 illustrates a detection system 100 for detecting a cross-reactive antibody 14 specific for the heavy chain of the target antibody 12. FIG. 3 illustrates a detection system 200 for detecting a cross-reactive antibody 16 specific for the light chain of the target antibody 12. FIG. 4 illustrates a detection system 300 for detecting a cross-reactive antibody 18 specific for an idiotypic portion of the target antibody 12.

The target antibody 12 in each of these detection systems can include a target antibody according to any of the embodiments described above. The detection systems also include a heavy chain detection antibody 24 and/or a light chain detection antibody 26. The heavy chain detection antibody 24 is labeled with a label 20 and the light chain detection antibody is labeled with another label 22. The labels 20, 22 can be any known labels in the art and in some cases are enzymatic, fluorescent or chemiluminescent labels. Also, in some cases the labels 20, 22 are different labels and in other cases the labels 20, 22 are the same label. Further, in some cases, the detection systems include both a heavy chain detection antibody 24 and a light chain detection antibody 26. In other cases, only one of the heavy chain detection antibody 24 or the light chain detection antibody 26 is used. The detection antibodies 24, 26 bind to the cross-reactive antibody 14, 16, 18.

In some embodiments, the detection antibodies 24, 26 are antibodies that detect isotypic portions of the heavy chains and light chains of the cross-reactive antibody. In other embodiments, the detection antibodies 24, 26 are antibodies that detect idiotypic and antigen-specific portions of the cross-reactive antibody. In some cases, the detection antibodies 24, 26 are non-mammalian antibodies that detect non-vertebrate antigens (e.g., keyhole limpet hemocyanin) on the cross-reactive antibodies. This way, the detection antibodies 24, 26 will not cross-react with any mammalian antibody.

In some embodiments, a heavy chain detection antibody 24 is used. In certain cases, the heavy chain detection antibody 24 is a cy5-phycoerythrin-labeled anti-human heavy chain specific antibody. In other embodiments, a light chain detection antibody 26 is used. In certain cases, the light chain defection antibody 26 is a phycoerythrin-labeled anti-human kappa light chain specific antibody or a phycoerythrin-labeled anti-human lambda light chain specific antibody.

Other embodiments of the invention provide for a method of detecting a cross-reactive antibody in a biological sample. The method includes steps of providing a biological sample 80 and providing a plurality of capture systems 40. The capture systems 40 can be capture systems according to any of the embodiments described above.

The method also includes adding or mixing the capture systems 40 to the biological sample 80. In some cases, the method includes mixing the capture systems 40 with the biological sample 80 for a period of time sufficient to allow binding of cross-reactive antibodies to target antibodies 12. In many cases, the period of time is between about 5 minutes and about 2 hours. In some cases, the period of time is between about 15 minutes and about 30 minutes. The method can also include subjecting the mixture to conditions that maximize the binding of cross-reactive antibodies to the target antibodies 12. In some cases, the mixture is mixed for a period of time using an orbital mixer. In other cases, the mixture is incubated for a period of time.

After this period of time expires, the method includes removing the capture systems 40 (along with any cross-reactive antibody bound to the target antibody 12) from the biological sample. In some cases, the method includes centrifuging the mixture to separate the capture systems 40. In other cases, the target particles 30 include paramagnetic particles and the method includes magnetically sequestrating the mixture 90 to separate the capture systems 40.

Next, the detection method includes washing the separated capture systems 40 to remove unbound cross-reactive antibodies. In some cases, the capture systems 40 are washed with PBS. After washing, the method includes mixing heavy chain specific detection antibodies 24 and/or a light specific chain detection antibodies 26 with the capture systems 40. During mixing, the detection antibodies 24, 26 bind to cross-reactive antibodies captured by the capture system 40. This mixing can also take place for a period of time sufficient to allow binding of heavy chain specific detection antibodies 24 and/or light chain specific detection antibodies 26 to the cross-reactive antibodies. In some cases, the period of time is between about 5 minutes and about 2 hours. In certain cases, the period of time is between about 15 minutes and about 30 minutes. The detection method can also include subjecting the mixture to conditions that maximize the binding of detection antibodies 24, 26 to cross-reactive antibodies. In some cases, the mixture is mixed for a period of time using an orbital mixer. In other cases, the mixture is incubated for a period of time.

Once the detection antibodies 24, 26 are bound to the cross-reactive antibodies (and thus to the capture systems), the capture systems are then washed to remove unbound detection antibodies 24, 26. The washed capture systems 40 are then examined using any known label detection system for detecting antibodies. In some cases, the detection antibodies are fluorescently-labeled antibodies and the detection method includes a fluorescent microscopy method.

If there are any detection antibodies 24, 26 detected, those detection antibodies 24, 26 are bound to cross-reactive antibodies 14, 16, 18, which are in turn bound to the target antibody 12. The detection antibodies 24, 26 therefore detect the presence of the cross-reactive antibodies. In certain cases, the detection antibodies are fluorescently labeled and then fluoresce with intensity proportional to the concentration of cross-reactive antibodies present in the biological sample.

### EXAMPLES

### Example 1

Example 1 describes one exemplary method of making biotinylated antibodies. In Example 1, monoclonal antibodies directed against keyhole limpet hemocyanin were diluted in 100 mM NaHCO₃ at a concentration of 5 mg/ml and then mixed with the N-hydroxysuccidimyl ester of biotin at a molar ratio of 10 moles biotin:1 mole antibody. Free biotin was separated from the conjugated antibody by size exclusion chromatography using Sephadex G-25.

### Example 2

Example 2 describes one exemplary method of making a target composition comprising capture systems. In Example 2, Applicant obtained paramagnetic target particles coated with avidin from Spherotech in Libertyville, Ill. Applicant washed the target particles in PBS and suspended them at a 1% w/v concentration (“particle suspension”). Applicant then added biotinylated antibodies to the particle suspension at a concentration of 60 micrograms per ml of particle suspension. Applicant next mixed the mixture with an orbital mixer for a period of time of one hour in order maximize binding of the biotinylated antibodies to the avidin coated paramagnetic particles to form the capture systems. Applicant then washed the capture systems with PBS to remove all unbound biotinylated antibodies. Applicant next re-suspended the capture systems in PBS at a concentration of about 3 million target particles/ml.

### Example 3

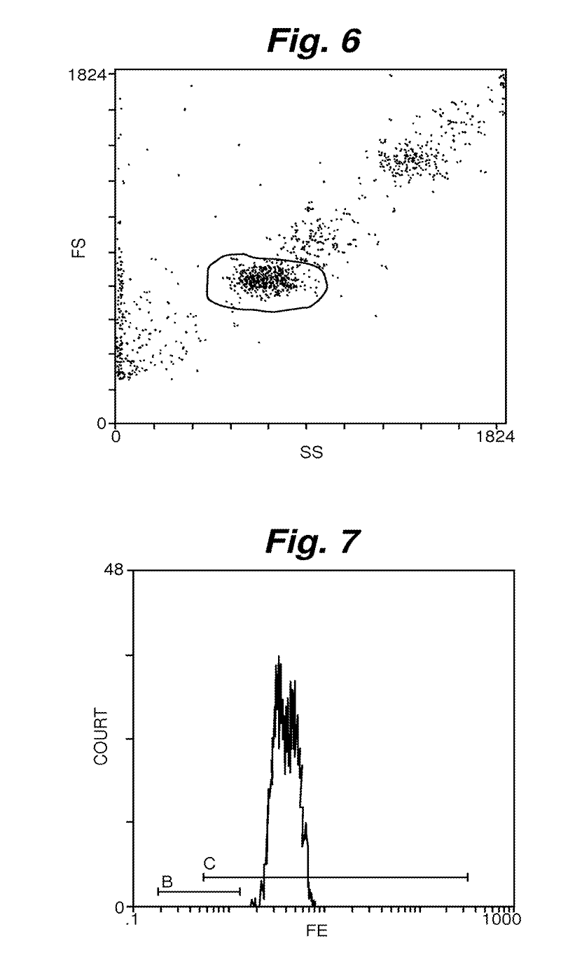

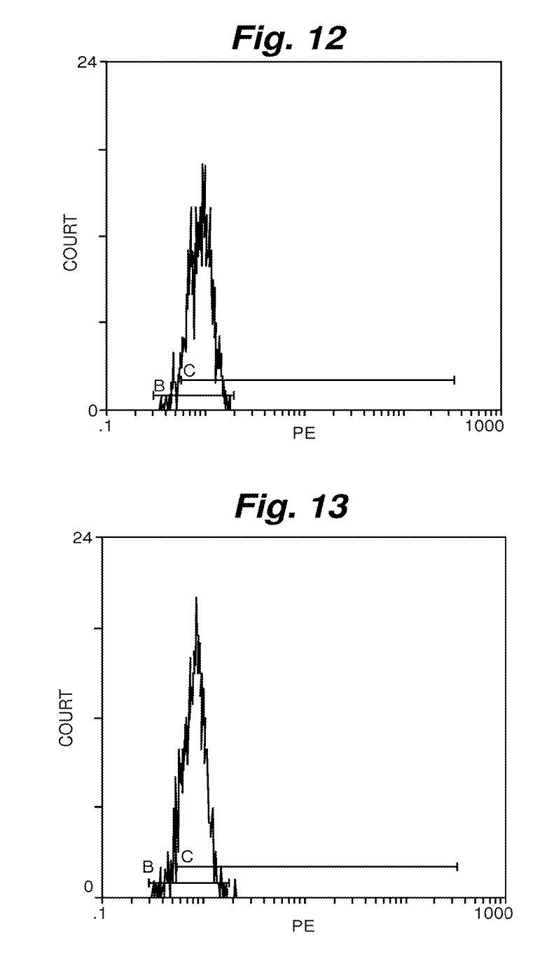

Example 3 shows a scatter plot in FIG. 6 showing light scatter patterns of paramagnetic target particles. The scatter plot was obtained by flow cytometric analysis and shows physical properties of the paramagnetic target particles. The y-axis shows particle size and the x-axis shows particle complexity. As shown, the paramagnetic target particles are composed of a single population light scatter-wise as shown by the cluster of dots surrounded by the little circle. This scatter plot can be used to gate for fluorescence detection only on the particles within that circle. This is the initial gating parameter that was performed on every histogram shown FIGS. 7-13.

### Example 4

Example 4 illustrates the specificity of one embodiment of the detection system. In Example 4, Applicant obtained a commercially available serum sample that had been identified as containing cross-reactive HAMA OKT3 antibodies. Applicant also obtained a target composition containing capture systems in PBS at a concentration of about 3 million target particles/ml. The capture systems included mouse monoclonal OKT3 target antibodies. Applicant then mixed 10 microliters of the target composition with 100 microliters of the serum sample. Applicant then placed the mixture on an orbital mixer for a period of time of between 15 to 30 minutes at room temperature. After mixing, Applicant separated the capture systems and washed them to remove unbound cross-reactive HAMA OKT3 antibodies. Applicant next mixed the capture systems with 100 to 500 ng of phycoerythrin labeled monoclonal anti-human kappa antibodies and allowed the mixture to sit for a period of time between about 15 to 30 minutes at room temperature. Applicant next washed the capture systems with PBS to remove unbound phycoerythrin labeled monoclonal anti-human kappa antibodies. Applicant then analyzed the capture systems using a standard flow cytometric analysis. FIG. 7 illustrates a single parameter fluorescence histogram of the flow cytometric analysis. The single parameter histogram shows that there was reactivity of the cross-reactive HAMA OKT3 antibodies with the mouse monoclonal OKT3 target antibodies and detected with phycoerythrin labeled monoclonal anti-human kappa antibodies.

### Example 5

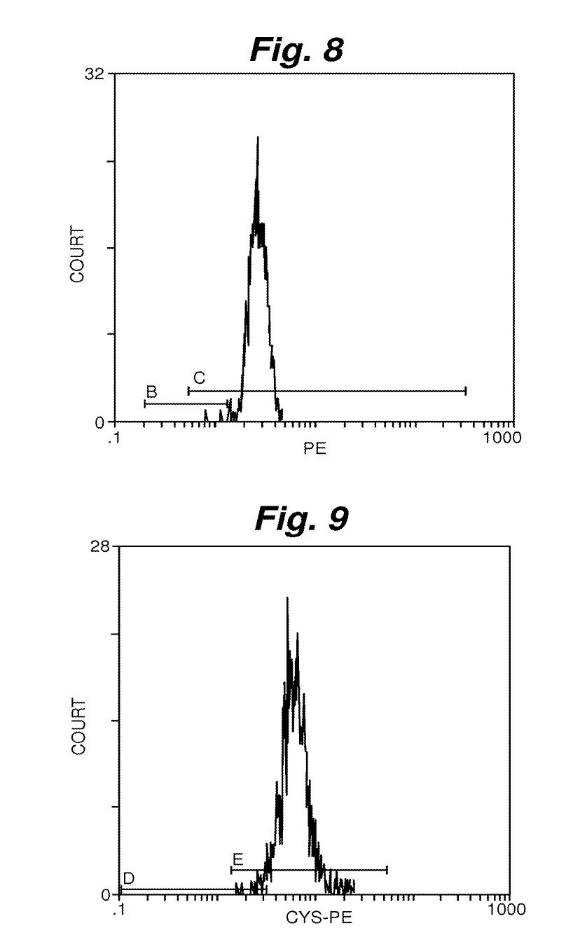

Example 5 illustrates the specificity of another embodiment of the detection system. In Example 5, Applicant obtained a commercially available serum sample that had been identified as containing cross-reactive HAMA OKT3 antibodies. Applicant also obtained a target composition containing capture systems in PBS at a concentration of about 3 million target particles/ml. The capture systems included mouse monoclonal OKT3 target antibodies. Applicant then mixed 10 microliters of the target composition with 100 microliters of the serum sample. Applicant then placed the mixture on an orbital mixer for a period of time of between 15 to 30 minutes at room temperature. After mixing, Applicant separated the capture systems and washed them to remove unbound cross-reactive HAMA OKT3 antibodies. Applicant next mixed the capture systems with 100 to 500 ng of phycoerythrin labeled monoclonal anti-human lambda antibodies and allowed the mixture to sit for a period of time between 15 to 30 minutes at room temperature. Applicant next washed the capture systems with PBS to remove unbound phycoerythrin labeled monoclonal anti-human lambda antibodies. Applicant then analyzed the capture systems using a standard flow cytometric analysis. FIG. 8 illustrates a single parameter histogram of the flow cytometric analysis. The single parameter histogram shows that there was reactivity of the cross-reactive HAMA OKT3 antibodies with the mouse monoclonal OKT3 target antibodies and detected with phycoerythrin labeled monoclonal anti-human lambda antibodies.

### Example 6

Example 6 also illustrates the specificity of another embodiment of the detection system. In Example 6, Applicant obtained a commercially available serum sample that had been identified as containing cross-reactive HAMA OKT3 antibodies. Applicant also obtained a target composition containing capture systems in PBS at a concentration of about 3 million target particles/ml. The capture systems included mouse monoclonal OKT3 target antibodies. Applicant then mixed 10 microliters of the target composition with 100 microliters of the serum sample. Applicant then placed the mixture on an orbital mixer for a period of time of between 15 to 30 minutes at room temperature. After mixing, Applicant separated the capture systems and washed them to remove unbound cross-reactive HAMA OKT3 antibodies. Applicant next mixed the capture systems with 100 to 500 ng of cy5-phycoerythrin labeled goat anti-human polyclonal antibodies (heavy and light chain reactive) and allowed the mixture to sit for a period of time of between 15-30 minutes at room temperature. Applicant next washed the capture systems with PBS to remove unbound cy5-phycoerythrin labeled goat anti-human polyclonal antibodies. Applicant then analyzed the capture systems using a standard flow cytometric analysis. FIG. 9 illustrates a single parameter histogram of the flow cytometric analysis. The single parameter histogram shows that there was reactivity of the cross-reactive HAMA OKT3 antibodies with the mouse monoclonal OKT3 target antibodies and detected with cy5-phycoerythrin labeled goat anti-human polyclonal antibodies.

### Example 7

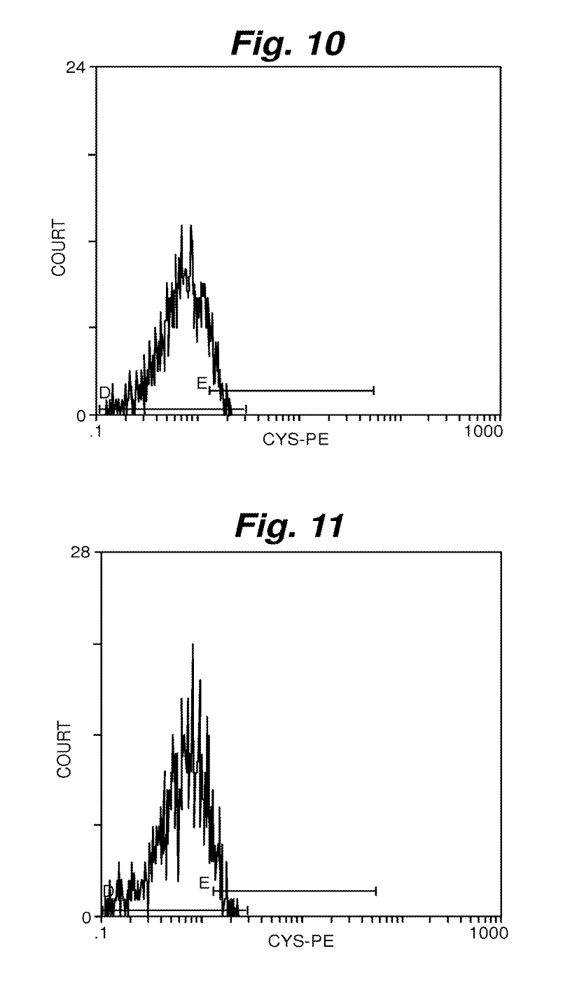

In Example 7, Applicant obtained a commercially available serum sample that had been identified as containing cross-reactive HAMA OKT3 antibodies. Applicant also obtained a target composition containing capture systems in PBS at a concentration of about 3 million target particles/ml. The capture systems included mouse monoclonal IgM target antibodies. Applicant then mixed 10 microliters of the target composition with 100 microliters of the serum sample. Applicant then placed the mixture on an orbital mixer for a period of time of between 15-30 minutes at room temperature. After mixing, Applicant separated the capture systems and washed them to remove unbound cross-reactive HAMA OKT3 antibodies. Applicant next mixed the capture systems with 100 to 500 ng of cy5-phycoerythrin labeled goat anti-human polyclonal antibodies (heavy and light chain reactive) and allowed the mixture to set for a period of time between 15-30 minutes at room temperature. Applicant next washed the capture systems with PBS to remove unbound cy5-phycoerythrin labeled goat anti-human polyclonal antibodies. Applicant then analyzed the capture systems using a standard flow cytometric analysis. FIG. 10 illustrates a single parameter histogram of the flow cytometric analysis. The single parameter histogram shows that there was a lack of reactivity of the cross-reactive HAMA OKT3 antibodies (mouse IgG2a isotype) with the mouse monoclonal IgM target antibodies.

### Example 8

In Example 8, Applicant obtained a commercially available serum sample that had been identified as containing cross-reactive HAMA OKT3 antibodies. Applicant also obtained a target composition containing capture systems in PBS at a concentration of about 3 million target particles/ml. The capture systems included mouse monoclonal IgG1 target antibodies. Applicant then mixed 10 microliters of the target composition with 100 microliters of the serum sample. Applicant then placed the mixture on an orbital mixer for a period of time of between 15-30 minutes at room temperature. After mixing, Applicant separated the capture systems and washed them to remove unbound cross-reactive HAMA OKT3 antibodies. Applicant next mixed the capture systems with 100 to 500 ng of cy5-phycoerythrin labeled goat anti-human polyclonal antibodies (heavy and light chain reactive) and allowed the mixture to sit for a period of time between 15-30 minutes at room temperature. Applicant next washed the capture systems with PBS to remove unbound cy5-phycoerythrin labeled goat anti-human polyclonal antibodies. Applicant then analyzed the capture systems using a standard flow cytometric analysis. FIG. 11 illustrates a single parameter histogram of the flow cytometric analysis. The single parameter histogram shows that there was a lack of reactivity of the cross-reactive HAMA OKT3 antibodies (mouse IgG2a isotype) with the mouse monoclonal IgG1 target antibodies.

### Example 9

In Example 9, Applicant obtained a commercially available serum sample that had been identified as containing cross-reactive HAMA OKT3 antibodies. Applicant also obtained a target composition containing capture systems suspended in PBS at a concentration of about 3 million target particles/ml. The capture systems included mouse monoclonal OKT3 target antibodies. Applicant added 100 microliters of the target composition to a test tube and placed the target composition in a magnetic field to sequester the capture systems. The separated PBS fluid was then removed by pipette. Applicant next added 1 ml of serum sample to the test tube. The test tube was then placed on an orbital mixer for 15-30 minutes at room temperature. After mixing, Applicant again placed the test tube in a magnetic field to separate the capture systems from the serum sample. Applicant used a pipette to remove the serum sample. Applicant also added to the serum sample capture systems that included paramagnetic target particles coated with avidin and bound to mouse monoclonal OKT3 target antibodies labeled with biotin. Applicant then incubated the mixture for a period of time. The capture systems were added in an amount necessary to remove the cross-reactive HAMA OKT3 antibodies from the serum sample. After incubation, Applicant separated the capture systems and removed them. Applicant then mixed the capture systems with phycoerythrin labeled monoclonal anti-human kappa antibodies. Applicant then washed the capture systems to remove unbound phycoerythrin labeled monoclonal anti-human kappa antibodies and analyzed the capture systems using a standard flow cytometric analysis. FIG. 11 illustrates a single parameter histogram of the flow cytometric analysis of the serum sample post HAMA removal. The single parameter histogram shows a removal of the cross-reactive HAMA OKT3 antibodies as the detection beads are now negative.

### Example 10

In Example 10, Applicant obtained a commercially available serum sample that had been identified as containing cross-reactive HAMA OKT3 antibodies. Applicant also added to the serum sample capture systems that included paramagnetic target particles coated with avidin and bound to mouse monoclonal OKT3 target antibodies labeled with biotin. Applicant then incubated the mixture for a period of time. The capture systems were added in an amount necessary to remove the cross-reactive HAMA OKT3 antibodies from the serum sample. After incubation, Applicant separated the capture systems and removed them. Applicant then mixed the capture systems with phycoerythrin labeled monoclonal anti-human lambda antibodies. Applicant then washed the capture systems to remove unbound phycoerythrin labeled monoclonal anti-human lambda antibodies and analyzed the capture systems using a standard flow cytometric analysis. FIG. 13 illustrates a single parameter histogram of the flow cytometric analysis. The single parameter histogram shows a removal of the cross-reactive HAMA OKT3 antibodies as reflected by removal of fluorescence.

In the foregoing detailed description, the invention has been described with reference to specific embodiments. However, it may be appreciated that various modifications and changes can be made without departing from the scope of the invention. When the claims refer to a component, skilled artisans will understand that the component can also be interpreted to be a plurality of the components. For example, the term “capture system” can be construed to include a single capture system or a plurality of capture systems.

What is claimed is:

1. A method of removing a cross-reactive antibody from a biological sample, the method comprising the steps of:
providing capture systems each comprising a target particle and a target antibody, wherein the target antibody is bound to the target particle;
providing a biological sample, wherein the biological sample comprises a cross-reactive antibody;
adding the capture systems to the biological sample, wherein the target antibody binds to the cross-reactive antibody;
removing the capture systems from the biological sample, wherein the cross-reactive antibody is bound to the target antibody and the target antibody is bound to the target particle.

2. The method of claim 1 wherein the target particle is a target particle coated with avidin or streptavidin and the target antibody is labeled with biotin, wherein the avidin or streptavidin binds to biotin.

3. The method of claim 1 wherein the target particle is a paramagnetic particle and the step of removing the capture systems from the biological sample comprises placing the biological sample in a magnetic field to separate the capture systems from the biological sample and then removing the separated capture systems.

4. The method of claim 1 wherein the removing the capture system from the biological sample comprises placing the biological sample in a centrifuge to separate the capture systems from the biological sample and then removing the separated capture systems.

5. The method of claim 1 wherein the target antibody is an animal antibody and the desired cross-reactive antibody is a human anti-animal antibody.

6. The method of claim 5 wherein the animal antibody is a mouse antibody and the desired cross-reactive antibody is a human anti-mouse antibody.

7. A method, comprising:
providing an immunoassay that uses an immunoassay antibody;
providing a capture system comprising a target particle and a target antibody, wherein the target antibody is bound to the target particle;
providing a biological sample, wherein the biological sample comprises a cross-reactive antibody that reacts with the immunoassay antibody;
adding the capture system to the biological sample, wherein the target antibody binds to the cross-reactive antibody;
removing the capture system from the biological sample, wherein the removing the capture system also removes the cross-reactive antibody bound to the target antibody and wherein the biological sample becomes substantially free of the cross-reactive antibody; and
using the biological sample in an immunoassay.

8. The method of claim 7 wherein the target antibody is of the same isotype as the immunoassay antibody.

9. The method of claim 7 wherein the target antibody is of the same species as the immunoassay antibody.

10. The method of claim 7 wherein the target antibody is the same as the immunoassay antibody.

11. The method of claim 7 wherein the immunoassay antibody and the target antibody are animal antibodies and the cross-reactive antibody is a human anti-animal antibody.

12. The method of claim 11 wherein the immunoassay antibody and the target antibody are mouse antibodies and the cross-reactive antibody is a human anti-mouse antibody.

13. The method of claim 7 wherein the target particle is a paramagnetic particle and the step of removing the capture system from the biological sample comprises of placing the biological sample in a magnetic field to separate and then remove the capture system.

14. A method of identifying a cross-reactive antibody from a biological sample, the method comprising the steps of:
providing a capture system, the capture system comprising a target particle and a target antibody, wherein the target antibody is bound to the target particle;
providing a biological sample, wherein the biological sample comprises a cross-reactive antibody;
adding the capture system to the biological sample, wherein the target antibody binds to the cross-reactive antibody;
removing the capture system from the biological sample, wherein the cross-reactive antibody is bound to the target antibody and the target antibody is bound to the target particle;
mixing a detection antibody with the capture system, wherein the detection antibody binds to the cross-reactive antibody;
analyzing the capture system to detect the detection antibody, wherein the detection antibody is bound to the cross-reactive antibody and the cross-reactive antibody is bound to the target antibody and the target antibody is bound to the target particle, and wherein detection of the detection antibody indicates presence of the cross-reactive antibody.

15. The method of claim 14 wherein the target particle is a target particle coated with avidin or streptavidin and the target antibody is labeled with biotin, wherein the avidin or streptavidin binds to biotin.

16. The method of claim 14 wherein the target particle is a paramagnetic particle and the step of removing the capture system from the biological sample comprises placing the biological sample in a magnetic field to separate the capture system from the biological sample and then removing the separated capture system.

17. The method of claim 14 wherein the target antibody is an animal antibody and the desired cross-reactive antibody is a human anti-animal antibody.

18. The method of claim 17 wherein the animal antibody is a mouse antibody and the desired cross-reactive antibody is a human anti-mouse antibody.

19. A method of preparing a composition that removes cross-reactive antibodies from a biological sample, the method comprising the steps of:
coating a target particle with avidin or streptavidin;
labeling a target antibody with biotin;
mixing the target particle with the target antibody for a period of time to allow the avidin or streptavidinin to bind to the biotin, which causes the target particle to bind to the target antibody; and
suspending the bound target particle and target antibody in a suspension fluid.
